(19) United States
(12) Patent Application Publication
(10) Pub. No.: US 2025/0179246 A1
(43) Pub. Date: Jun. 5, 2025

(54) A LIQUID-LIQUID EXTRACTION METHOD FOR SEPARATING CARBOXYLATE MONOFUNCTIONAL PERFLUOROPOLYETHERS AND CARBOXYLATE DIFUNCTIONAL PERFLUOROPOLYETHERS

(71) Applicant: ZHEJIANG UNIVERSITY, HANGZHOU, Zhejiang Province (CN)

(72) Inventors: XILI CUI, HANGZHOU, ZHEJIANG PROVINCE (CN); HUABIN XING, HANGZHOU, ZHEJIANG PROVINCE (CN); LIFENG YANG, HANGZHOU, ZHEJIANG PROVINCE (CN); XIAN SUO, HANGZHOU, ZHEJIANG PROVINCE (CN)

(21) Appl. No.: 18/843,696

(22) PCT Filed: Jun. 21, 2023
(86) PCT No.: PCT/CN2023/101824

§ 371 Date: Sep. 3, 2024

(30) Foreign Application Priority Data
Jun. 23, 2022 (CN) 202210726209.0

(51) Int. Cl. C08G 65/30; B01D 11/04; C07C 51/48; C07C 59/135

(52) U.S. Cl. CPC C08G 65/30; B01D 11/0492; C07C 51/48; C07C 59/135

(57) ABSTRACT

The invention discloses a liquid-liquid extraction method for separating carboxylate monofunctional perfluoropolyethers and carboxylate difunctional perfluoropolyethers, which comprises: the mixture containing monocarboxylate end-group perfluoropolyether and dicarboxylate end-group perfluoropolyether are mixed in contact with light and heavy two-phase solvent, after extraction equilibrium, the two phases were separated and the solvent is removed, and the high-purity monocarboxylate end-group perfluoropolyether is obtained in heavy phase and dicarboxylate end-group perfluoropolyether is obtained in light phase. The light phase solvent comprises a diluent and a polar solvent containing fluorine atoms. The diluent is one or more of sulfolane, dimethyl sulfoxide, N-methylpyrrolidone, N,N-dimethylformamide, water, ethylene glycol, acetonitrile, acetic acid, ethanol, methanol. A heavy phase solvent consists of a non-polar solvent containing fluorine atoms and a polar solvent containing fluorine atoms, or a non-polar solvent containing fluorine atoms and a non-polar solvent without fluorine atoms.

### TECHNICAL FIELD

The present invention relates to the technical field of separation of monofunctional and difunctional perfluoropolyether active compounds, in particular to a liquid-liquid extraction method for separating carboxylate monofunctional perfluoropolyethers and carboxylate difunctional perfluoropolyethers.

### BACKGROUND OF TECHNOLOGY

Functionalized (per) fluoropolyether (PFPE-AC) active compounds are high-molecular-weight polymers with active terminal groups. The molecular backbone of PFPE-ACs consists only of C, F, and O atoms, endowing them with excellent thermal stability, chemical inertness, and low surface energy. The active terminal groups can be further modified, making these compounds suitable for applications in automotive, aerospace, microelectronics, chemical engineering, textile processing, and protective building coatings. However, functionalized PFPE-AC are typically mixtures containing monofunctional and difunctional PFPEs, which possess different properties. Therefore, it is crucial to purify PFPEs with the desired functionality for industrial applications. Currently, widely used PFPE-ACs have carboxylate terminal groups. The carboxylate monofunctional PFPEs can serve as precursors for PFPEs with other terminal groups, such as alcohol, ester, and amide groups, which can further be converted into PFPE derivatives (e.g., polyurethane derivatives, polyacrylate derivatives, and silanized derivatives). The production process of carboxylate monofunctional PFPEs may lead to contamination with non-functional and difunctional PFPEs mixtures, significantly affecting the subsequent processing and performance of the final products.

Currently, separation methods for such mixtures mainly involve physical or chemical adsorption and chromatography. A small number of methods also involve molecular distillation and chemical reaction-based separations. For example, patents CN104768623A and WO2014067981A1 describe using intermittent chromatography to separate mixtures containing inert perfluoropolyethers (PFPEs), monocarboxylate PFPE, and dicarboxylate PFPE, aiming to increase the average functionality of PFPE carboxylates. The process employs silica gel as an adsorbent, requiring multiple adsorption and elution steps. Patent CN105518054A by Daikin Industries uses thin-layer chromatography with silica gel to separate similar mixtures, involving multiple adsorptions and elutions with various non-polar and polar mobile phases. Patents JP2015164908A and JP2017222732A report adsorption separation techniques for isolating carboxylates from starting mixtures containing non-functional, monocarboxylate, and dicarboxylate PFPEs. Similarly, Shin-Etsu Chemical Corporation's patent EP2905298B1 uses supercritical CO₂ as a mobile phase and silica gel as a stationary phase for such separations. In patent WO2014067981A1, an intermittent chromatography method is used for the purification of perfluoropolyether carboxylates. This method can enhance the average functionality of the perfluoropolyether carboxylates. The separation process involves contacting a perfluoropolyether mixture with a solid phase, and through the separation of the solid and liquid phases, obtaining high-functionality perfluoropolyether carboxylates. In patent JP2015164906A, chromatography technology is employed to increase the proportion of mono-carboxylated perfluoropolyether compounds from a mixture of mono- and di-carboxylated perfluoropolyether compounds. This method uses supercritical or subcritical carbon dioxide as mobile phase and silica gel as solid phase for chromatographic analysis, but the separation method is complicated and does not allow the preparation of a large number of single-ended perfluoropolyethers containing carboxyl groups.

In summary, the current methods for separating mixtures of perfluoropolyether carboxylates with different functionalities primarily involve adsorption separation and chromatographic separation. However, these methods are relatively cumbersome, requiring multiple adsorption and elution steps. Consequently, they involve high solvent consumption and result in low yields.

### SUMMARY OF THE INVENTION

To address the aforementioned technical issues and deficiencies in the field, the present invention provides a liquid-liquid extraction method for separating carboxylate monofunctional perfluoropolyethers and carboxylate difunctional perfluoropolyethers. This method features simple operation, stable processes, high production capacity, high purity, high yield, and low cost.

The specific technical scheme is as follows:

A liquid-liquid extraction method for separating carboxylate monofunctional perfluoropolyethers and carboxylate difunctional perfluoropolyethers, comprising: the mixture containing monocarboxylate end-group perfluoropolyether and dicarboxylate end-group perfluoropolyether are mixed in contact with light and heavy two-phase solvent. After extraction equilibrium, the two phases were separated and the solvent was removed (distillation and other methods may be used), and the high-purity monocarboxylate end-group perfluoropolyether is obtained in heavy phase and dicarboxylate end-group perfluoropolyether is obtained in light phase.

The light phase solvent comprises a diluent and a polar solvent containing fluorine atoms. The diluent is one or more of sulfolane, dimethyl sulfoxide, N-methylpyrrolidone, N,N-dimethylformamide, water, ethylene glycol, acetonitrile, acetic acid, ethanol, methanol.

A heavy phase solvent consists of a non-polar solvent containing fluorine atoms and a polar solvent containing fluorine atoms, or a non-polar solvent containing fluorine atoms and a non-polar solvent without fluorine atoms.

The light-phase solvent described in this invention has a boiling range of 50-150° C., while the heavy-phase solvent has a boiling range of 60-180° C.

In a preferred embodiment, the content of carboxylate monofunctional perfluoropolyether in the mixture containing carboxylate monofunctional perfluoropolyether and carboxylate difunctional perfluoropolyether is 4 wt %˜96 wt %;

The monocarboxylate end-group perfluoropolyether has the structure shown in formula (I):

A-R_{f1}-B₁ (I)

The dicarboxylate end-group perfluoropolyether has the structure shown in formula (II):

B₂₁-R_{f2}-B₂₂ (II)
wherein:

A=F or C1-C4 perfluoroalkyl;
B₁, B₂₁ and B₂₂ are all carboxylate functional groups and are independently selected.
R_{f1} and R_{f2} are main chains of perfluoropolethers with an average molecular weight of 300˜25000 meet the chemical structure shown in equation (3) below, and they are independently selected:
R_{f1} and R_{f2} are perfluoropolyether main chains with number average molecular weights ranging from 300 to 25,000, both of which satisfy the chemical structure shown in the following formula (III) and are independently selected:

—O(CF₂O)ₐ—(C₂F₄O)_{b}—(C₃F₆O)_{c}—(C₄F₈O)_{d}— (III)
wherein:
a, b, c, and d are independent integers ranging from 0 to 300, and the sum of a, b, c, and d is greater than 3. The repeating units (CF₂O), (C₂F₄O), (C₃F₆O), (C₄F₈O) exist in any order, and the repeating units (C₃F₆O) include one or more of the following structures: (CF₂CF₂CF₂O), (CF₂CF(CF₃)O) and (CF(CF₃)CF₂O).

The carboxylate functional groups in carboxylate monofunctional perfluoropolyethers and carboxylate difunctional perfluoropolyethers are each independently selected, and the expressions all satisfy —CFXCOOM, wherein:
X=F, propenyl, methylpropenyl, one of the C₁-C₄ alkyls in which all or part of the hydrogen atoms are replaced by fluorine atoms;
M=Na⁺, K⁺, R_{b}⁺, or Cₛ⁺.

The polar solvents containing fluorine atoms are one or more of carboxylic acids containing fluorine atoms, ketones containing fluorine atoms and alcohols containing fluorine atoms.

The non-polar solvents containing fluorine atoms are one or more of hydrofluoroalkanes, unsaturated halogenated hydrocarbons, perfluoroalkanes, perfluorinated or partially fluorinated aromatic solvents, fluorinated ethers, fluorinated esters, and perfluorinated amines.

The non-polar solvents that do not contain fluorine atoms are one or more of hydrocarbon solvents, hydrocarbon monoethers, and aromatic solvents.

The non-polar solvent that does not contain fluorine atoms is preferably a solvent that is compatible with the non-polar solvent containing fluorine atoms.

The content of diluent in light phase solvent is 5 wt %˜45 wt %, preferably 6 wt %˜40 wt %, the content of polar solvent containing fluorine atom is 55 wt %˜95 wt %, preferably 60 wt %˜94 wt %;

In a preferred embodiment, the heavy-phase solvent consists of a non-polar solvent containing fluorine atoms and a polar solvent containing fluorine atoms.

Further preferably, the heavy phase solvent consists of a non-polar solvent containing fluorine atoms and a polar solvent containing fluorine atoms, and the content of the non-polar solvent containing fluorine atoms is 60 wt %˜95 wt %, and the content of the polar solvent containing fluorine atoms is 5 wt %˜40 wt %;

The liquid-liquid extraction is one or more combinations of kettle extraction, cross-flow extraction, counter-current extraction and fractionation extraction.

During the extraction process, the light-phase solvent and the heavy-phase solvent separate into upper and lower phases. If the ratio of light-phase solvent to heavy-phase solvent is too low or too high, it can lead to increased solvent usage or poor extraction efficiency. Therefore, the ratio must be determined based on the distribution coefficients of the mono-functional (per) fluoropolyether carboxylate compounds and the di-functional (per) fluoropolyether carboxylate compounds in each solvent phase system. The ratio of light phase solvent to heavy phase solvent is 0.2˜6:1.

Temperature affects the phase equilibrium relationship of mono-functional (per) fluoropolyether carboxylate compounds and di-functional (per) fluoropolyether carboxylate compounds in the two-phase solvents. Appropriately increasing the extraction operation temperature can accelerate the mass transfer rate between the two phases, while ensuring that the operation temperature remains below the boiling point of the selected solvents. The extraction temperature is 10˜70° C., preferably 15˜65° C.

The liquid-liquid extraction method for separating carboxylate monofunctional perfluoropolyethers and carboxylate difunctional perfluoropolyethers is characterized in that the purity of monocarboxylate end-group perfluoropolyether is not less than 98%, the yield is not less than 75%.

The beneficial effects of the present invention are as follows:
1. For the first time, the present invention employs liquid-liquid extraction technology for the separation of mono- and di-functionalized perfluoropolyethers with carboxylate end groups. The extraction process is simple, the workflow is straightforward, the operation is stable, the production capacity is high, and the costs are relatively low.
2. The solvents used in the present invention are easy to recover.
3. The separation products of the present invention have high purity, enabling the isolation of mono-functional perfluoropolyether carboxylates with a purity of no less than 98% from mixtures containing (per) fluoropolyether carboxylates, while also achieving high yield.

### DETAILED DESCRIPTIONS OF THE EMBODIMENTS

Some illustrative but not limitative Examples of the present invention follow. In each of the following Examples, “%” is based on a molar basis, if not otherwise indicated.

### Example 1

Z-type perfluoropolyther mixture (3 g) containing 60% single-ended sodium carboxylate and 40% double-ended sodium carboxylate was mixed with 120 ml pentafluoroacetone, 12 ml N,N-dimethylformamide and 100 ml NOVEC® HFE 7200. Extraction was carried out at 30° C., phase separation was placed, extraction liquid and raffinate were collected, solvent was removed after concentration. Monofunctional perfluoropolyether sodium carboxylate with purity of 98.5% was prepared with yield of 85%.

### Example 2

Z-type perfluoropolyther mixture (3 g) containing 45% single-ended sodium carboxylate and 55% double-ended sodium carboxylate was mixed with 130 ml trifluoroacetic acid, 40 ml N,N-dimethylformamide, 10 ml ethanol and 110 ml tetradecafluorohexane. Extraction was carried out at 25° C., phase separation was placed, extraction liquid and raffinate were collected, solvent was removed after concentration. Monofunctional perfluoropolyether sodium carboxylate with purity of 98.3% was prepared with yield of 80%.

### Example 3

Z-type perfluoropolyther mixture (3 g) containing 80% single-ended potassium carboxylate 20% double-ended potassium carboxylate was mixed with 130 ml and 1,1,1,3,3,3-Hexafluoro-2-propanol, 35 ml sulfolane and 110 ml 1,1,2,2,3,3,4-heptafluorocyclopentane. Extraction was carried out at 20° C., phase separation was placed, extraction liquid and raffinate were collected, solvent was removed after concentration. Monofunctional perfluoropolyether potassium carboxylate with purity of 98.7% was prepared with yield of 83%.

### Example 4

Y-type perfluoropolyther mixture (3 g) containing 75% single-ended potassium carboxylate and 25% double-ended potassium carboxylate was mixed with 150 ml 2,2,2-Trifluoroethanol, 55 ml dimethyl sulfoxide and 100 ml NOVEC® HFE 7300. Extraction was carried out at 35° C., phase separation was placed, extraction liquid and raffinate were collected, solvent was removed after concentration. Monofunctional perfluoropolyether potassium carboxylate with purity of 98.9% was prepared with yield of 87%.

### Example 5

Z-type perfluoropolyther mixture (3 g) containing 70% single-ended sodium carboxylate and 30% double-ended sodium carboxylate was mixed with 150 ml pentafluoro-1-propanol, 40 ml dimethyl sulfoxide and 100 ml AE-3000. Extraction was carried out at 30° C., phase separation was placed, extraction liquid and raffinate were collected, solvent was removed after concentration. Monofunctional perfluoropolyether sodium carboxylate with purity of 98% was prepared with yield of 93%.

### Example 6

Y-type perfluoropolyther mixture (3 g) containing 50% single-ended potassium carboxylate and 50% double-ended potassium carboxylate was mixed with 50 ml trifluoroacetic acid, 30 ml dimethyl sulfoxide, 100 ml pentafluoro-1-propanol and 130 ml 1H,1H, 1H-pentadecafluoro-2-nonanone. Extraction was carried out at 20° C., phase separation was placed, extraction liquid and raffinate were collected, solvent was removed after concentration. Monofunctional perfluoropolyether potassium carboxylate with purity of 98.2% was prepared with yield of 90%.

### Example 7

Y-type perfluoropolyther mixture (3 g) containing 80% single-ended potassium carboxylate and 20% double-ended potassium carboxylate was mixed with 150 ml pentafluoro-1-propanol, 20 ml N,N-dimethylformamide, 20 ml water and 150 ml 2,2-Bis(3,4-dimethylphenyl) hexafluoropropane. Extraction was carried out at 30° C., phase separation was placed, extraction liquid and raffinate were collected, solvent was removed after concentration. Monofunctional perfluoropolyether potassium carboxylate with purity of 99% was prepared with yield of 89%.

### Example 8

Y-type perfluoropolyther mixture (3 g) containing 30% single-ended potassium carboxylate and 70% double-ended potassium carboxylate was mixed with 150 ml pentafluoro-1-propanol, 40 ml dimethyl sulfoxide, 20 ml methanol and 150 ml 1,1,1,2,2,3,4,5,5,5-decafluoropentane. Extraction was carried out at 50° C., phase separation was placed, extraction liquid and raffinate were collected, solvent was removed after concentration. Monofunctional perfluoropolyether potassium carboxylate with purity of 98.2% was prepared with yield of 92%.

### Example 9

K-type perfluoropolyther mixture (3 g) containing 50% single-ended potassium carboxylate and 50% double-ended potassium carboxylate was mixed with 100 ml pentafluoro-1-propanol, 30 ml N,N-dimethylformamide, 10 ml water and 150 ml NOVEC® HFE 7100. Extraction was carried out at 40° C., phase separation was placed, extraction liquid and raffinate were collected, solvent was removed after concentration. Monofunctional perfluoropolyether potassium carboxylate with purity of 98.8% was prepared with yield of 75%.

### Example 10

D-type perfluoropolyther mixture (3 g) containing 50% single-ended sodium carboxylate and 50% double-ended sodium carboxylate was mixed with 120 ml 2,2,2-trifluoroethanol, 40 ml N,N-dimethylformamide and 150 ml NOVEC® HFE 7100. Extraction was carried out at 40° C., phase separation was placed, extraction liquid and raffinate were collected, solvent was removed after concentration. Monofunctional perfluoropolyether sodium carboxylate with purity of 98.2% was prepared with yield of 90%.

It should also be understood that after reading the above description of the invention, persons skilled in the art may make various changes or modifications to the invention, and these equivalent forms also fall within the limits of the claims attached to this application.

1. A liquid-liquid extraction method for separating carboxylate monofunctional perfluoropolyethers and carboxylate difunctional perfluoropolyethers comprising the steps of:
mixing a mixture containing monocarboxylate end-group perfluoropolyether and dicarboxylate end-group perfluoropolyether in contact with a light phase solvent and a heavy phase solvent,
after extraction equilibrium, separating a light phase and a heavy phase and removing the solvents, and
obtaining the high-purity monocarboxylate end-group perfluoropolyether in the heavy phase and obtaining dicarboxylate end-group perfluoropolyether in the light phase;
wherein the light phase solvent comprises a diluent and a polar solvent containing fluorine atoms, the diluent is one or more of sulfolane, dimethyl sulfoxide, N-methylpyrrolidone, N,N-dimethylformamide, water, ethylene glycol, acetonitrile, acetic acid, ethanol, methanol;
The heavy phase solvent consists of a non-polar solvent containing fluorine atoms and a polar solvent containing fluorine atoms, or a non-polar solvent containing fluorine atoms and a non-polar solvent without fluorine atoms.

2. The liquid-liquid extraction method for separating carboxylate monofunctional perfluoropolyethers and carboxylate difunctional perfluoropolyethers according to claim 1, wherein content of carboxylate monofunctional perfluoropolyether in the mixture containing carboxylate monofunctional perfluoropolyether and carboxylate difunctional perfluoropolyether is 4 wt %˜96 wt %;
the monocarboxylate end-group perfluoropolyether has the structure shown in formula (I):
A-R_{f1}-B₁ (I)
the dicarboxylate end-group perfluoropolyether has the structure shown in formula (II):
B₂₁-R_{f2}-B₂₂ (II)
wherein:
A is F or C1-C4 perfluoroalkyl;
B₁, B₂₁ and B₂₂ are independently selected from all carboxylate functional groups;
R_{f1} and R_{f2} are main chains of perfluoropolethers with an average molecular weight of 300˜25000 meet the chemical structure shown in equation (III) below, and they are independently selected:
R_{f1} and R_{f2} are perfluoropolyether main chains with number average molecular weights ranging from 300 to 25,000, both of which satisfy the chemical structure shown in the following formula (III) and are independently selected:
—O(CF₂O)ₐ—(C₂F₄O)_{b}—(C₃F₆O)_{c}—(C₄F₈O)_{d}— (III)
wherein:
a, b, c, and d are independent integers ranging from 0 to 300, and the sum of a, b, c, and d is greater than 3. The repeating units (CF₂O), (C₂F₄O), (C₃F₆O), (C₄F₈O) exist in any order, and the repeating units (C₃F₆O) include one or more of the following structures: (CF₂CF₂CF₂O), (CF₂CF(CF₃)O) and (CF(CF₃)CF₂O).

3. The liquid-liquid extraction method for separating carboxylate monofunctional perfluoropolyethers and carboxylate difunctional perfluoropolyethers according to claim 1, wherein carboxylate functional groups in carboxylate monofunctional perfluoropolyethers and carboxylate difunctional perfluoropolyethers are each independently selected, and the expressions all satisfy-CFXCOOM, wherein:
X is F, propenyl, methylpropenyl, one of the C₁-C₄ alkyls in which all or part of the hydrogen atoms are replaced by fluorine atoms;
M=Na⁺, K⁺, R_{b}⁺, or Cₛ⁺.

4. The liquid-liquid extraction method for separating carboxylate monofunctional perfluoropolyethers and carboxylate difunctional perfluoropolyethers according to claim 1, wherein that the polar solvents containing fluorine atoms are one or more of carboxylic acids containing fluorine atoms, ketones containing fluorine atoms and alcohols containing fluorine atoms.

5. The liquid-liquid extraction method for separating carboxylate monofunctional perfluoropolyethers and carboxylate difunctional perfluoropolyethers according to claim 1, wherein non-polar solvents containing fluorine atoms are one or more of hydrofluoroalkanes, unsaturated halogenated hydrocarbons, perfluoroalkanes, perfluorinated or partially fluorinated aromatic solvents, fluorinated ethers, fluorinated esters, and perfluorinated amines.

6. The liquid-liquid extraction method for separating carboxylate monofunctional perfluoropolyethers and carboxylate difunctional perfluoropolyethers according to claim 1, wherein non-polar solvents that do not contain fluorine atoms are one or more of hydrocarbon solvents, hydrocarbon monoethers, and aromatic solvents.

7. The liquid-liquid extraction method for separating carboxylate monofunctional perfluoropolyethers and carboxylate difunctional perfluoropolyethers according to claim 1, wherein the content of diluent in light phase solvent is 5 wt %˜45 wt %, preferably 6 wt %˜40 wt %, the content of polar solvent containing fluorine atom is 55 wt %˜95 wt %, preferably 60 wt %˜94 wt %;
the heavy phase solvent consists of a non-polar solvent containing fluorine atoms and a polar solvent containing fluorine atoms, and the content of the non-polar solvent containing fluorine atoms is 60 wt %˜95 wt %, and the content of the polar solvent containing fluorine atoms is 5 wt %˜40 wt %;
the ratio of light phase solvent to heavy phase solvent is 0.2˜6:1.

8. The liquid-liquid extraction method for separating carboxylate monofunctional perfluoropolyethers and carboxylate difunctional perfluoropolyethers according to claim 1, wherein liquid-liquid extraction is one or more combinations of kettle extraction, cross-flow extraction, counter-current extraction and fractionation extraction.

9. The liquid-liquid extraction method for separating carboxylate monofunctional perfluoropolyethers and carboxylate difunctional perfluoropolyethers according to claim 1, wherein the extraction temperature is 10˜70° C., preferably 15˜65° C.

10. The liquid-liquid extraction method for separating carboxylate monofunctional perfluoropolyethers and carboxylate difunctional perfluoropolyethers according to claim 1, wherein the purity of monocarboxylate end-group perfluoropolyether is not less than 98%, the yield is not less than 75%.
